(19) United States
(12) Patent Application Publication
(10) Pub. No.: US 2010/0145194 A1
(43) Pub. Date: Jun. 10, 2010

(54) HISTOGRAM-BASED ANALYSIS METHOD FOR THE DETECTION AND DIAGNOSIS OF NEURODEGENERATIVE DISEASES

(75) Inventors: Abhinay D. Joshi, Drexel Hill, PA (US); John G. Wolodzko, Yardley, PA (US); Krishnendu Saha, Philadelphia, PA (US)

Correspondence Address: PEPPER HAMILTON LLP, ONE MELLON CENTER, 50TH FLOOR, 500 GRANT STREET, PITTSBURGH, PA, 15219, US

(73) Assignee: AVID RADIOPHARMACEUTICALS, INC., Philadelphia, PA (US)

(21) Appl. No.: 12/617,606

(22) Filed: Nov. 12, 2009

Related U.S. Application Data
(60) Provisional application No. 61/114,277, filed on Nov. 13, 2008.

(51) Int. Cl. A61B 6/00; G06K 9/00

(52) U.S. Cl. 600/431; 382/128; 382/131; 382/172

(57) ABSTRACT

An analysis method using histograms derived from positron emission tomography (PET) or single photon emission tomography (SPECT) images of the brain, which utilizes radiopharmaceuticals for the detection and diagnosis of pathological targets associated with neurodegenerative disease in a patient is provided.

This application claims priority under 35 U.S.C. §119(e) to U.S. Provisional Patent Application Ser. No. 61/114,277, filed Nov. 13, 2008, the disclosure of which is incorporated by reference in its entirety.

### BACKGROUND OF THE INVENTION

1. Field of the Invention

The present invention relates generally to methods for monitoring physiological activity in the brain and more specifically to the use of histograms representing brain scan image data for the detection of neurodegenerative diseases.

2. Description of Related Art

Alzheimer's disease (AD) is a progressive neurodegenerative disorder characterized by cognitive decline, irreversible memory loss, disorientation, and language impairment. AD affects 10% of the population aged greater than 65 and at least 50% of the population aged greater than 85 years. AD has been reported in patients as young as 40-50 years of age, but because the presence of the disease is difficult to detect without histopathological examination of brain tissue, the time of onset in living subjects is unknown.

Currently, the only means of definitively diagnosing AD is through examination of brain tissue, typically performed at postmortem autopsy. During the autopsy, medical examiners inspect the brain tissue for excess neuritic plaques (NPs) composed of amyloid-β peptide deposits and neurofibrillary tangles (NFTs) formed of filaments of highly phosphorylated tau proteins, as these features distinguish the pathogenesis of AD. The amyloid deposits are formed by aggregation of amyloid peptides, followed by further combination with other aggregates and/or amyloid peptides. The fibrillar aggregates of amyloid peptides, Aβ1-40 and Aβ1-42, are the major peptide metabolites derived from amyloid precursor protein that are found in NPs and cerebrovascular amyloid deposits in AD patients.

Parkinson's disease (PD) is a progressive neurodegenerative disease characterized by resting tremors, bradykinesia, muscular rigidity, and postural instability. PD typically develops after the age of 60, though 15% of diagnosed patients are under the age of 50. Family history of PD is an etiological factor for 5-10% of patients diagnosed with the disease, yet only 1% of cases have been shown to be clearly familial. It is estimated that 1.5 million Americans are currently living with PD.

Dementia with Lewy Bodies (DLB) is a progressive brain disease having symptoms that fluctuate between various degrees of manifestation. These symptoms include progressive dementia, Parkinsonian movement difficulties, hallucinations, and increased sensitivity to neuroleptic drugs. As with AD, advanced age is considered to be the greatest risk factor for DLB, with average onset usually between the ages of 50-85. Further, 20% of all dementia cases are caused by DLB and over 50% of PD patients develop “Parkinson's Disease Dementia” (PDD), a type of DLB. It is possible for DLB to occur alone, or in conjunction with other brain abnormalities, including those involved in AD and PD, as mentioned above. At present, a conclusive diagnosis of DLB is only possible after postmortem autopsy.

PD and DLB share an etiology of dopamine deficiency that is correlated with the death of dopaminergic neurons in the substantia nigra. The cause of dopaminergic neuronal death in PD is uncertain, although it appears that aggregates of α-synuclein in the brain may be associated with dopaminergic neuronal losses in the striatum. It is also recognized that in DLB, abnormal protein deposits containing α-synuclein, referred to as “Lewy bodies”, are the cause of the death of dopaminergic neurons. Lewy bodies occur mostly in the substantia nigra and locus ceruleus sections of the brain stem, and also in the subcortical and cortical regions of the brain. Because of this particular localization in the brain, Lewy bodies may interfere with the production of acetylcholine, causing disruption of perception and thought process and impacting behavior. Lewy bodies are considered to be a type of neuritic plaque (NP) because they are comprised of aggregates of α-synuclein protein deposits.

The etiology of neurodegeneration can also involve a mixture of pathologies including a component of microvascular, or perfusion, deficits in the brain. For example, a disorder commonly referred to as “mixed dementia” often comprises both perfusion deficits and amyloid plaque pathology. The term “mixed dementia” possesses various meanings, but the term is commonly used to refer to the coexistence of AD and vascular dementia (VaD), in particular where the VaD is caused by numerous micro-thrombi in the vascular system of the brain. Though little is currently known about the true prevalence of mixed dementia, this form of neurodegeneration is clinically important because the combination of AD and VaD may have a greater impact on the brain than either condition independently. Mixed dementia is traditionally very difficult to diagnose. Symptoms are similar to those of AD or VaD or a combination of the two.

The occurrence of amyloid deposits in the brain may be characteristic of numerous other conditions including, but not limited to, Mediterranean fever, Muckle-Wells syndrome, idiopathetic myeloma, amyloid polyneuropathy, amyloid cardiomyopathy, systemic neuritic amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, and islets of Langerhans diabetes Type II insulinoma.

Based on the numerous potential and often times overlapping causes of dementia and other types of neurodegenerative diseases, current clinical diagnostic tools and methods must be enhanced to improve the sensitivity and specificity for the detection and monitoring of the underlying pathology or pathologies in neurodegenerative diseases, particularly those due to Aβ aggregates in AD and α-synuclein deposits in PD and DLB.

### SUMMARY OF THE INVENTION

It is an object of the present invention to provide methodology for creating histograms of PET and SPECT scans of pathologic targets in a patient's brain. Another object of the invention is to utilize histogram analysis to diagnose neurodegenerative diseases, including Alzheimer's disease (AD). A further object of the invention is to utilize the histograms to estimate the total amount of pathologic target (e.g., Aβ or α-synuclein aggregates) in the brain, the highest relative concentration of the pathologic target in the brain, and the frequency distribution of various concentrations of the pathologic target across the entire brain or across specific volume planes of the brain.

These objects are achieved in accordance with one aspect of the invention by a method for detecting the presence of a pathologic target in a brain of a patient including administering to the patient a radiopharmaceutical capable of binding to a pathologic target in the brain of the patient, obtaining image data of at least a portion of the cranium of the patient, generating a histogram from the image data, and analyzing a feature of the histogram to detect the presence of the pathologic target in the patient's brain.

In some embodiments, the method further includes the step of eliminating from the image data radioactive signal from outside of the patient's brain. In this embodiment, the presence of two or more peaks or a dispersed distribution of elevated higher intensity bin counts over a segment of the histogram is indicative of the presence of the pathologic target in the patient's brain, while the presence of one peak representing relatively lower intensity radioactivity in the patient's cranium is indicative of the absence of significant pathologic target in the patient's brain.

In aspects of the invention where the histogram represents the entire image volume from the image data of the cranium, the presence of two peaks representing relatively lower intensity radioactivity in the patient's cranium compared to a histogram for a comparative patient having a significant amount of the pathologic target in the comparative patient's cranium is indicative of the absence of significant pathologic target in the patient's brain, while the presence of more than two peaks or a dispersed distribution of elevated higher intensity bin counts over a segment of the histogram is indicative of the presence of the pathologic target in the patient's brain.

In another aspect of the invention, a method for the detection or measurement of a pathologic target in a brain of a patient is provided that includes administering to a patient a radiopharmaceutical capable of binding to a pathologic target in the brain of the patient, obtaining image data of the brain of the patient, generating a histogram from the image data, wherein the histogram includes intensity versus frequency curves resulting from different populations of binding sites of the radiopharmaceutical in the patient's brain, and applying a mathematical method of separating or deconvoluting the intensity versus frequency curves.

In yet another aspect of the invention, a method is provided for predicting the relative risk for future development of a dementia such as, for example, Alzheimer's disease (AD), Dementia with Lewy Bodies (DLB), or Vascular dementia (VaD) in a subject, where the method comprises administering to the subject one or more radiopharmaceuticals that bind to aggregates of β-amyloid, α-synuclein, tau protein or micro-thrombi in the subject's brain, obtaining image data of the subject's brain, generating an intensity versus frequency histogram from volume elements of the image data, and evaluating at least one of shape, area, peak value and other graphical parameters of the histogram to detect the presence of the aggregates of β-amyloid, α-synuclein, tau protein or micro-thrombi in the subject's brain, wherein the absence of a higher intensity peak in the histogram representing specific binding of the radiopharmaceutical to the pathologic target within the subject's brain is indicative of lower risk of future development of the dementia, and the presence of the higher intensity peak in the histogram representing the specific binding of the radiopharmaceutical to the pathologic target within the subject's brain is indicative of a relatively higher risk for the presence or future development of the dementia.

The above summary of the present invention is not intended to describe each illustrated aspect or every implementation of the present invention. The figures and the detailed description that follow particularly exemplify these aspects.

### BRIEF DESCRIPTION OF THE FIGURES

For a better understanding of the disclosure, reference should be made to the following figures, in which:

FIG. 1A is a PET brain scan image of ¹⁸F-AV-45 in a healthy control subject in transverse, coronal, and sagittal orientation;

FIG. 1B is a histogram depicting the activity inside and outside of the brain of the healthy control subject of FIG. 1A;

FIG. 2A is a PET brain scan image of ¹⁸F-AV-45 in an Alzheimer's disease (AD) patient in transverse, coronal, and sagittal orientation;

FIG. 2B is a histogram depicting the activity inside and outside of the brain of the Alzheimer's disease (AD) patient of FIG. 2A;

FIG. 3A is a PET brain scan image of ¹⁸F-AV-45 in an elderly subject having increased levels of Aβ aggregates in transverse, coronal, and sagittal orientation;

FIG. 3B is a histogram depicting the activity inside and outside of the brain of the subject shown in FIG. 3A;

FIG. 4A is a histogram generated from image voxel elements of higher intensity within an amyloid-negative patient's brain after excluding background radioactivity outside the brain;

FIGS. 4B-D are histograms generated from image voxel elements of higher intensity within different target-positive patients' brains after excluding background radioactivity outside the brains;

FIG. 5A is a histogram derived from the whole image of an amyloid PET scan of a subject without amyloid pathology in the brain; and

FIG. 5B is a histogram derived from the same image utilized in FIG. 5A, after removal of an area of skull and soft tissues from histogram processing.

FIG. 6 is a histogram and first derivative profile of one embodiment of the present invention; and

FIG. 7 is a graph illustrating the ratio of area after a histogram inflection point relative to the area before the inflection point according to one embodiment of the invention.

### DESCRIPTION OF ILLUSTRATIVE EMBODIMENTS

This invention is not limited to the particular compositions or methodologies described, as these may vary. In addition, the terminology used in the description describes particular versions or embodiments only and is not intended to limit the scope of the invention. Unless defined otherwise, all technical and scientific terms used herein have the same meanings as commonly understood by one of ordinary skill in the art. In case of conflict, the patent specification, including definitions, will prevail.

As used herein, the singular forms “a”, “an” and “the” include plural reference unless the context clearly dictates otherwise.

As used herein, the term “about” means plus or minus 10% of the numerical value of the number described. Therefore, about 50% is in the range of 45%-55%.

“Administering” when used in conjunction with an diagnostic agent, such as, for example, a radiopharmaceutical, means to administer directly into or onto a target tissue or to administer the radiopharmaceutical systemically to a patient whereby the diagnostic agent is used to image the tissue or a pathology associated with the tissue to which it is targeted. “Administering” a composition may be accomplished by injection, infusion, or by either method in combination with other known techniques.

The terms “comprise”, “have” and “include” and their conjugates, as used herein, mean “including, but not necessarily limited to.”

“Histogram”, as used herein, refers to a graph or plot that is generated from the number of volume elements (e.g., voxels) in an image containing values of a specified intensity range. An example of the histogram described herein is a plot of signal intensity on the x-axis versus the number of voxels (i.e., volume elements) containing that signal intensity on the y-axis (or vice-versa), where the signal intensity may be expressed as Becquerels per voxel or as a Standardized Uptake Value (SUV), as well as other units of radioactivity per volume known to those skilled in the art. The frequency may be expressed as the number of volume elements (i.e., voxels) in that range of signal intensity.

As used herein, the terms “healthy control”, “healthy control subject”, or “healthy subject” refers to a patient not exhibiting the clinical signs or symptoms of the specific neurodegenerative disorder being evaluated. Healthy controls may include those individuals who have a pathologic or aberrant protein, peptide or polynucleotide present in the brain, yet still do not exhibit signs or symptoms of the clinical disease (e.g., AD or DLB).

As used herein, the term “pathologic target” refers to a compound associated with dementia, VaD, AD, DLB, PDD or other neurodegenerative disease. Examples of pathologic targets include an abnormal concentration of a native or pathologically-altered protein, peptide or oligonucleotide; β-amyloid; α-synuclein; phosphorylated-tau protein aggregates; Lewy bodies; neurofibrillary tangles; micro-thrombi in the vascular system of a patient's brain; a tumor cell-associated antigen in a patient's brain; an antigen that is upregulated due to inflammation in a patient's brain and an antigen which is upregulated due to vascular disease in a patient's brain.

The term “pathology”, as used herein, refers to an altered biological process, which may be associated with the aberrant production of proteins, peptides, RNA, and other substances associated with the disease process, or in some instances may be associated with losses of endogenous markers expressed in normal tissue due to the pathology (e.g., losses of nigrostriatal dopaminergic neurons in Parkinson's disease (PD)).

As used herein, the terms “patient” and “subject” refer to any living organism to which the compounds described herein are administered and whose brain activity is to be measured in conjunction with performing the analysis methods of the invention. Patients and/or subjects may include, but are not limited to, any non-human mammal, primate or human. Such patients and/or subjects may or may not be exhibiting signs, symptoms or pathology of one or more particular diseased states.

As used herein, the term “radiopharmaceutical” refers to a compound or material which is suitable for administration to humans and has attached thereto one or more radioactive atoms emitting photons that may be detected outside the body utilizing devices such as, but not limited to, for example, positron emission tomography (PET) or single photon emission tomography (SPECT) cameras.

As used herein, the term “scan volume” refers to a three dimensional volume in which a radiopharmaceutical in the head (i.e., cranium) is measured. The scan volume is typically divided into an array of voxels.

The term “standardized uptake value” (SUV), as used herein, is a representation of radioactivity concentrations in a given image region relative to the injected tracer amount. The SUV is a dimensionless figure calculated by taking the ratio of local radioactivity concentration (in kilobecquerels per gram of tissue) to the administered amount of radiopharmaceutical per gram body weight (in kilobecquerels per gram of tissue).

The term “target” when used in conjunction with a diagnostic agent, such as a radiopharmaceutical, refers to tissue or other material associated with pathology to which localization of the radiopharmaceutical or diagnostic agent is desired. Targets may include, but are not limited to, diseased cells, pathogens, infectious material or other undesirable material in a patient, such as abnormal proteins, peptides, RNA or DNA.

Generally speaking, as used herein, the term “tissue” refers to any aggregation of similarly specialized cells that are united in the performance of a particular function.

As used herein, the term “volume of interest” refers to one or more specific voxels of the brain of a patient. The volume of interest can be inclusive of a two dimensional or three dimensional object.

The term “voxel”, as used herein, generally refers to a volume element comprising a three dimensional volume from which one or more measurements are made. A voxel may be a single measurement unit or may be part of a larger three dimensional grid array that covers a volume.

Various embodiments of the invention are directed to a method for analyzing brain scan data of a patient for neurodegenerative disease, dementia or cognitive impairment, such as Alzheimer's disease (AD), Parkinson's disease (PD), Dementia with Lewy Bodies (DLB), and Vascular Dementia (VaD), using histograms.

In one embodiment of the invention, methodology is provided for creating histograms from positron emission tomography (PET) or single photon emission tomography (SPECT) scans of pathologic targets in a patient's brain. In another embodiment, a histogram is utilized to diagnose neurodegenerative disease, including, for example, Alzheimer's disease (AD). In yet another embodiment of the invention, a histogram is utilized to estimate the total amount of pathologic target (e.g., Aβ or α-synuclein aggregates) in the brain of a patient, the highest relative concentration of pathologic target in the brain, and the frequency distribution of various concentrations of pathologic target across the entire brain or across specific volume planes of the brain in order to diagnose or monitor neurodegenerative disease in a patient.

According to certain aspects of the invention, a histogram tool included in various image display instruments and software utilized in the analysis of brain scan images is used to generate voxel versus numerical range bar graphs. These histograms may represent an entire image volume including the entire skull and surrounding area. The histograms may be analyzed or presented together with three dimensional images reconstructed from a PET or SPECT scan or may be analyzed or displayed without requiring any image processing or presentation and do not necessarily require any fundamental knowledge of brain anatomy to derive useful information regarding the quantity or concentration of a given pathologic target in the brain of a patient. The distributions of voxel values and the associated histogram shapes differ in a regular and repeatable fashion between the images of healthy control subjects and those of patients exhibiting the clinical or preclinical symptoms of AD or other neurodegenerative disease. As such, visual inspection of the histogram may be used to differentiate members of these two populations (i.e., normal and abnormal).

Various embodiments of the invention are directed to a method for detecting neurodegenerative disease including AD in a patient by evaluating histograms of image data derived from the patient's brain after administration of a β-amyloid specific radiopharmaceutical. Such methods are based on the observation that histograms representing brain scan data of patients exhibiting clinical or subclinical signs of AD or dementia reveal a distinctly different shape than histograms of healthy control patients. That is, by simply inspecting the shapes of the resulting histogram plots, it is possible to diagnose AD pathology.

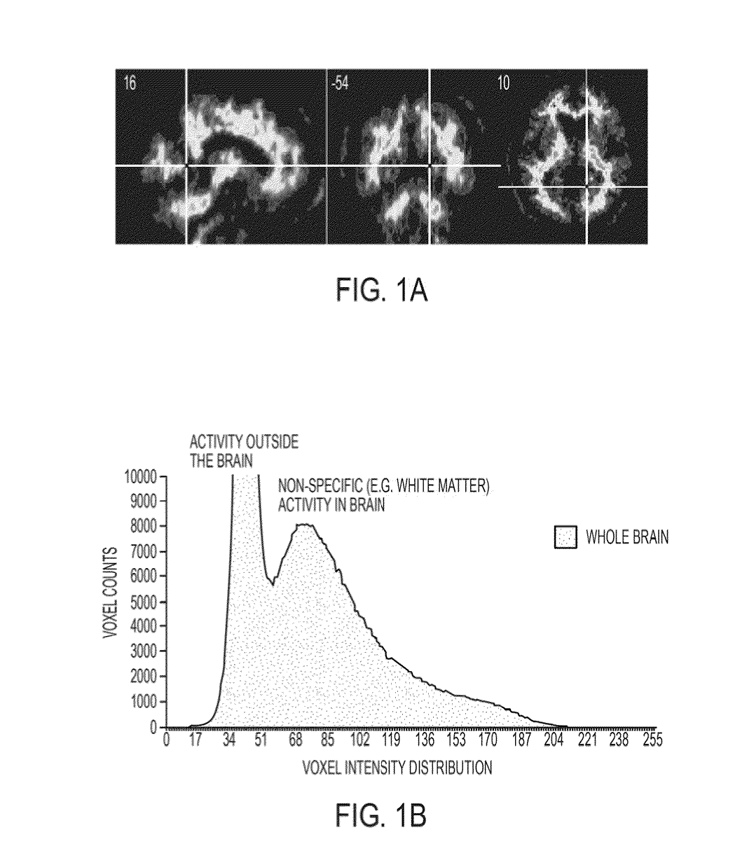

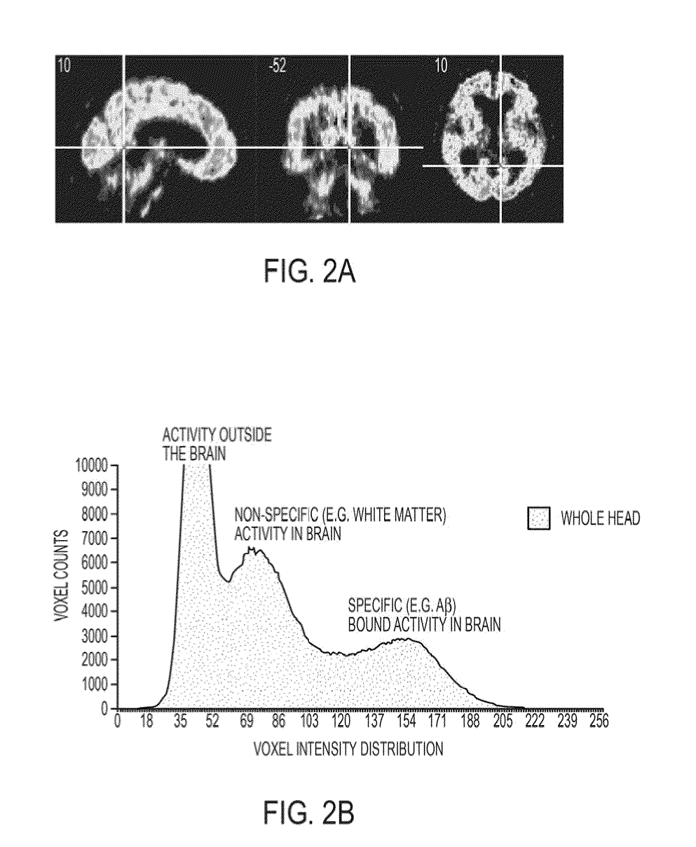

In still other aspects of the invention, a method for detecting the presence of a pathologic target in a brain of a patient is provided comprising administering to the patient one or more imaging agents capable of binding to the pathologic target, obtaining image data of the patient's brain, generating a histogram, and analyzing a feature of the histogram in order to detect the presence of the pathologic target in the patient's brain. In certain aspects of the invention, a histogram having two peaks representing radioactivity within a subject's cranium (one peak due to activity outside the brain and the other being a single peak representing non-specifically retained activity within the patient's brain) is indicative of the absence of significant pathologic target in the patient's brain. In another aspect of the current invention, a histogram of the brain image that shows more than two such peaks or a dispersed distribution of elevated bin counts over a segment of the histogram is indicative of the presence of pathologic target in the patient's brain (as shown in FIGS. 1B and 2B).

According to other aspects, a method for detecting neurodegenerative disease in a patient is provided comprising administering at least one radiopharniaceutical for detecting a pathologic target associated with a neurodegenerative disease to a patient, measuring the distribution of radioactivity of the radiopharmaceutical in a portion of the patient comprising a region of the patient wherein the pathologic target associated with the neurodegenerative disease is anticipated to be positioned, employing computer executable logic to manipulate the measured distribution of radioactivity of the radiopharmaceutical to generate a histogram, and analyzing the characteristics of the histogram to detect and quantitate the presence of the pathologic target associated with the neurodegenerative disease.

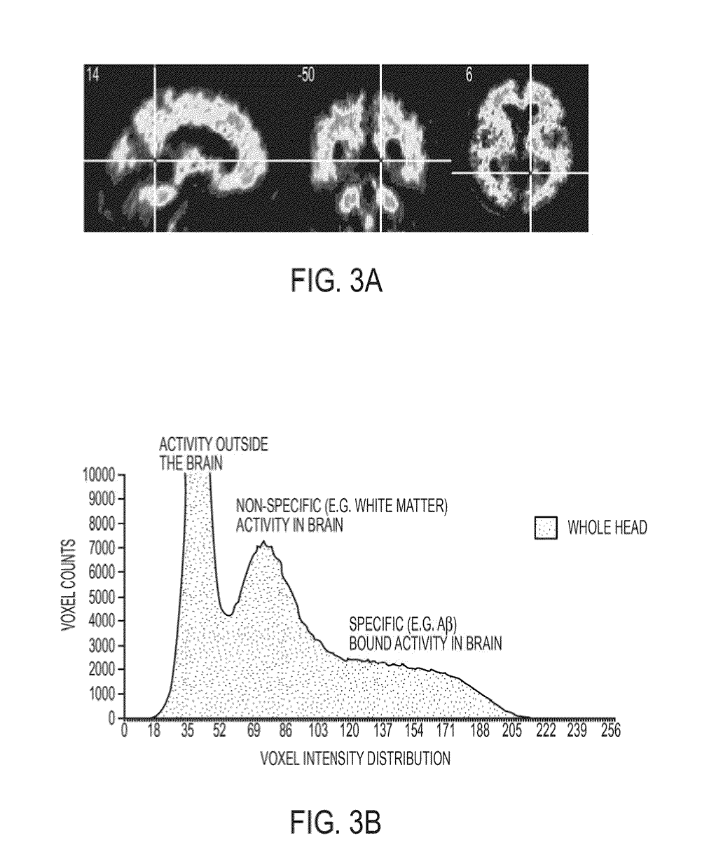

In yet another aspect of the invention, a method is provided for predicting the relative risk for the future development of neurodegenerative disease including AD, where the method comprises administering to the subject one or more radiopharmaceuticals that bind to Aβ-containing neuritic plaques in the patient's brain, obtaining image data of the patient's brain, generating a histogram, which represents the entire image volume from the image data, and analyzing at least one aspect of the shape, area, peak value, and related parameters of the histogram to detect the presence of the Aβ plaque in the patient's cranium, wherein a histogram having two peaks representing the radiopharmaceutical present within the patient's cranium is indicative of a lower risk of the future development of AD (as shown in FIG. 1B) and a histogram displaying three or more such peaks or a dispersed distribution of bin counts following the second peak of the histogram is indicative of the presence of Aβ plaque in the patient's brain and is indicative of a relatively higher risk for the future development of AD (as exemplified by FIG. 2B or FIG. 3B).

FIG. 1A shows a positron emission tomography (PET) brain scan image of ¹⁸F-AV-45 (a radiopharmaceutical that binds to Aβ aggregates) in a healthy control subject of age 83 and mini-mental state examination (MMSE) of 30 in transverse, coronal, and sagittal orientations, respectively. FIG. 1B is a histogram based on voxel number and voxel intensity, created from the cranial images of the healthy control subject in FIG. 1A depicting the activity inside and outside of the healthy control subject's brain. As shown in FIG. 1B, two distinct peaks are observed. A first “off-scale” peak represents voxel values from outside the brain that include, but are not limited to, background activity in the scalp, blood vessels, and facial bones. A second peak located to the right of the first peak represents voxel values or activity from inside the brain and includes volume elements having higher signal intensities due to non-specific binding of the radiopharmaceutical to the normal white and gray matter of the brain. Notably absent is a third peak of voxels having higher signal intensity. The absence of this third peak indicates that the subject of FIG. 1A does not have appreciable levels of pathologic Aβ aggregates in the gray matter of the brain.

FIG. 2A shows ¹⁸F-AV-45 PET brain scan images of an AD patient of age 78 and MMSE of 22 in transverse, coronal, and sagittal orientation, respectively. FIG. 2B is a histogram of voxel counts (number of volume elements of a given signal intensity) versus signal intensity created from the cranial images of the AD patient of FIG. 2A depicting the activity inside and outside of the brain of the patient. The histogram shown in FIG. 2B differs from the histogram of the imaging data of the healthy control subject shown in FIG. 1B. In particular, in FIG. 2B there is an observable third peak to the right of the second peak, which is not present in the histogram of the healthy control subject in FIG. 1B. As such, a comparison of voxel counts, intensity, and number of peaks in a histogram derived from brain scan images of a patient to a histogram derived from imaging data from a healthy control subject facilitates the diagnosis of AD pathology. Furthermore, integration of the third peak, if present in the histogram, is representative of the total amount of radiopharmaceutical bound to Aβ aggregates in the brain and, therefore, is proportional to the amount of pathologic target (Aβ aggregate) in the brain. In addition, the x-axis intercept of the maximum of the third peak of the histogram is representative of the highest concentration of radiopharmaceutical bound to Aβ aggregates in the brain, and therefore would be proportional to the highest concentration of pathologic target (Aβ aggregate) in the brain.

FIG. 3A shows a ¹⁸F-AV-45 PET brain scan of a patient of age 84 and MMSE 30 (cognitively normal) in transverse, coronal, and sagittal orientation, respectively. FIG. 3B is a histogram depicting the imaging data of the brain of the patient of FIG. 3A. Although the histogram depicted in FIG. 3B is derived from the images of a clinically healthy control subject, FIG. 3B differs from the histogram of the imaging data of the healthy control subject shown in FIG. 1B. In particular, an increase in voxel bin counts in a segment of the histogram to the right of the second peak is observable in FIG. 3B that is not present in the histogram depicted in FIG. 1B. The elevated voxel bin count in the FIG. 3B histogram reflects the uptake of the radiopharmaceutical in amyloid plaque and therefore may be an indicator of the potential for future development of AD or a prodromal form of AD. The characteristics of the histogram, in particular the segment of the histogram with elevated voxel bin counts indicating detectable aggregated Aβ in the gray matter of the brain, suggests that the patient of FIG. 3B may be at risk for future development of clinical AD, in contrast to the subject in FIG. 1B whose histogram does not show a third peak or segment of elevated (i.e., higher intensity) voxel bin counts.

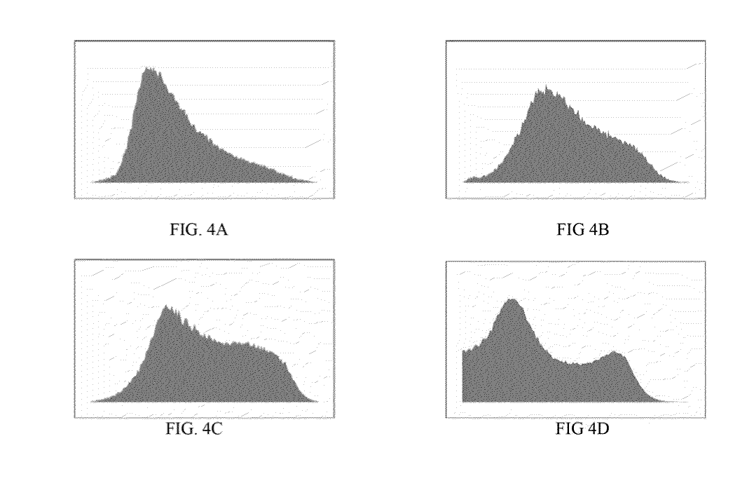

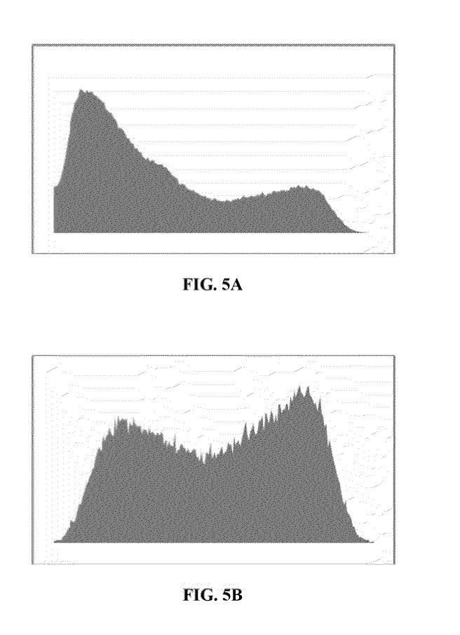

The histogram may reflect the total frequency distribution of voxel intensity in the image. However, as previously described, it is possible to create the histograms from only those image voxel elements of higher intensity within the brain itself (i.e., excluding the background radioactivity outside the brain). In this circumstance, in amyloid-negative subject brains as well as in an in-vitro phantom (e.g., Hoffmann phantom) there exists one peak population of voxels (or pixels) (FIG. 4A). In patients with higher radiopharmaceutical concentrations per voxel, due to pathology in the brain, there is an increase in the population of high intensity voxels (which may reflect specific binding to a target such as amyloid) that changes the histogram appearance. An additional arm (FIG. 4B), or plateau (FIG. 4C) or additional peak (FIG. 4D) on the histogram curve occurs in target-positive (e.g., pathology-positive) patients and reflects an increased target concentration in the brain that may clinically correspond to disease related pathology in the brain. The relative amount of high intensity voxels (or voxels) in the image increases significantly as areas of non-specific uptake (e.g., radioactivity outside the brain, or activity in white matter (e.g., non-pathologic) regions of the brain are excluded from the histogram analysis, which demonstrates that low intensity pixels in the histogram created from the image of the head corresponds to non-specific uptake and the presence of high intensity pixels are related to radiopharmaceutical binding in relatively higher concentration to the pathologic target of interest in the brain. For example, in a subject without amyloid pathology in the brain, a histogram derived from an amyloid PET scan will show that the second peak is more dominant in the histogram after the area of skull and soft tissues have been removed from the histogram processing (FIG. 5A and FIG. 5B represent histogram of the whole image and the same image after outside-brain-tissue removal, respectively).

In some embodiments, the image data analyzed is derived from an image encompassing the entire brain, skull and surrounding region (as shown in, for example, FIGS. 1B, 2B and 3B), and in other aspects, analysis is limited to one or more volumes of interest. Such volumes of interest may encompass any portion of a brain image including portions that include any lobe of the brain, white matter, gray matter, skull, intracranial space, brain stem, and medulla oblongata, to name a few. In certain embodiments, the image data may be manipulated to isolate a volume of interest. For example, the image may be cropped to remove portions of the brain not associated with the volume of interest and in some embodiments, the skull may be excluded from the image data analyzed, such as in, for example, FIGS. 4A, 4B and 4C. In such aspects of the present invention, the portion of the brain analyzed or portions of the image data excluded from analysis can be identified or selected automatically by, for example, including or excluding any portion of the image having voxel data over a predetermined intensity threshold. Alternatively, the histogram analysis may be performed on a section or slab of the brain that is prepared by reconstruction of the PET or SPECT image in a given plane of the brain.

In certain aspects of the present invention, histograms derived from analyzed brain images may be further manipulated to improve resolution of the peaks and to clarify or magnify differences between diseased brains and those of healthy control subjects. For example, a histogram derived from brain images may be fit using Gaussian fitting, exponential fitting, polynomial fitting, any other fitting algorithm known in the art or any combination thereof. The fitting of histograms utilized in various aspects of the invention may reveal additional features of the histograms not readily apparent by examination of the raw or unanalyzed histogram data, such as the area under peaks, peak intensity or the breadth of peaks. For example, the tracer (e.g., radiopharmaceutical) target content can be estimated by a method of histogram curve analysis.

Any mathematical description of the histogram curve capable of separating the two or more voxel populations in an image may be useful in semi-quantitative evaluation of PET or SPECT brain images. If different voxel populations are due to specific and non-specific uptake in the brain, mathematical processing may produce quantitative estimates of the specific radiopharmaceutical-pathologic-target binding based on its area under the histogram profile following the mathematical separation of the underlying frequency-intensity voxel populations. Mathematical techniques such as estimating change in curvature of the histogram profile based on first or second order derivatives or Fourier-transforms of the histogram provide methodology that can accurately estimate the separation of one or more populations of radiopharmaceutical binding populations within the image histogram profile.

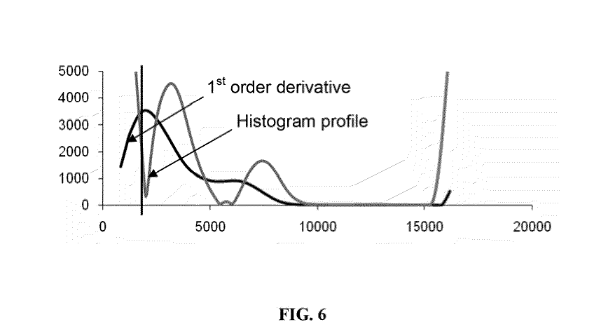

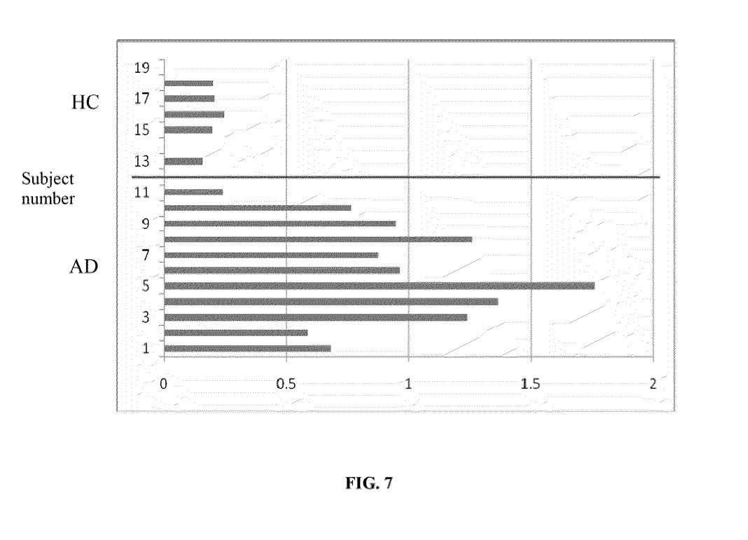

Use of mathematical curve separation tools in the analysis of image histograms facilitate disease diagnosis and prognosis as well as the monitoring of treatment effects and follow up of individual patients. As an example, in one embodiment of the invention, amyloid tracer images were analyzed in amyloid-pathology-negative and amyloid-pathology-positive patients. The detection of the inflection point on a descending slope of a histogram curve (based on a first derivative analysis) enabled division of the histogram into two parts, as shown in FIG. 6. FIG. 7 is a graph of the ratios of the areas under the histogram curve for post-inflection point (i.e. higher intensity specific radiopharmaceutical binding) relative to pre-inflection point (i.e. lower intensity non-specific radiopharmaceutical binding) voxel populations from the amyloid PET scans of cognitively normal and Alzheimer's disease subjects (red line separates both groups).

In some aspects of the invention, the image data undergoes additional analysis steps. For example, in one embodiment, image data derived from a PET or SPECT scan is inputted into a processor that identifies individual voxels or groups of voxels having brightness (i.e. intensity) greater than a predetermined threshold or an average background. This may be accomplished using curve-separation methods such as the derivative method illustrated in FIG. 5, or by other mathematical transforms well-known to those skilled in the art of curve stripping and analysis. The identified and separated higher intensity voxels in the image histogram can be correlated to the presence of radiopharmaceutical at or above a predetermined threshold signal level. In another embodiment, image data is derived from images that are scanned and inputted into a processor, which identifies bright spots on the image to determine radiopharmaceutical distribution. In yet another embodiment, analysis of the image data includes measuring the intensity, concentration or strength of the output brightness or any combination thereof. These measurements may correlate to the amount of radiopharmaceutical in the image, an area or region on the image or a particular spot on the image. An area or spot on an image having a greater intensity than other areas or spots may contain a higher concentration of radiopharmaceutical targeted to, for example, β-amyloid and, thus, the region where the β-amyloid localizes in the brain of a particular patient can be identified. Image data may also be analyzed specifically on spatial location of volumes of interest to which the administered radiopharmaceuticals are targeted (e.g., the regions of the brain where the disease pathology is known to be located). Without wishing to be bound by theory, by identifying areas, regions, spots or volumes of interest on an image that correlate to the presence of a radiopharmaceutical, a neurodegenerative disease or progression of such a neurodegenerative disease may be identified by the histogram method described herein.

Various embodiments of the invention are directed to methods in which one or more steps are completed before the histogram is produced. For example, the methods of the invention may include steps of creating the image, equilibrating the image, gray-scaling the image, reducing background, cropping the image, mapping the image, color coding the image, combining one or more images, overlaying one or more images, fusing one or more images, extracting voxel-by-voxel data, normalizing extracted voxel data or otherwise manipulating image data in a manner such that a histogram may be obtained.

The methods of various aspects of the invention may include the steps of administering one or more radiopharmaceuticals or other imaging agents targeting neuritic plaque, such as Aβ or α-synuclein aggregates, to a patient, obtaining image data of the patient's brain or head, generating a histogram from the image data, and analyzing the histogram. In such aspects, overall voxel count and shape of the histogram may be analyzed to diagnose the patient with AD, dementia or other neurodegenerative disease. In certain embodiments of the invention, the analysis can be performed through visual inspection where a histogram having two peaks representing radioactivity within the patient's cranium is indicative of the absence of high concentrations of a pathologic target (e.g., neuritic plaque) and a histogram displaying more than two peaks or a dispersed distribution of voxel bin counts over a segment of the histogram is indicative of the presence of higher concentrations of the pathologic target in the patient's brain. In some aspects of the invention, this determination can be made in the absence of comparative control data from a healthy subject.

Image data that serves as the basis for the histogram may be acquired by any method known in the art and the step of imaging and measuring the distribution of activity may be carried out by any procedure known in the art that permits imaging of one or more radiopharmaceuticals administered to a patient. For example, in certain embodiments of the invention, imaging is carried out by positron emission tomography (PET) or single photon emission tomography (SPECT) imaging. In other embodiments, imaging may be carried out by both PET and SPECT or by combined imaging methods such as PET/CT (PET with concurrent computed tomography imaging) or PET/MRI (PET with concurrent magnetic resonance imaging).

The PET and SPECT methodologies utilize activity measurements to determine radiopharmaceutical biodistributions throughout different regions of the subject brain scan images. The volumes of interest located in the brain scan images are analyzed relative to the anatomy of a standard reference brain to which the subject brain scan images are spatially registered and normalized. These analyses may be carried out using averages of standardized uptake values (SUVs) or other values measured within the volumes of interest. Software often used for brain image analysis includes MRIcroN, MATLAB® R2006B, Statistical Parametric Mapping (SPM), as well as Microsoft Excel, and combinations thereof. Results are typically presented as functional images representing color-coded SUV or SUV ratio maps of the brain.

PET scans can be analyzed in at least two general ways. The first general approach involves segmenting a PET scan of the brain into volumes of interest for each of several regions of interest (depending on the pathology or disease being evaluated). The volumes of interest are adjusted for radioactive decay to the time of radioactive tracer injection and SUVs are calculated. The SUVs in specific disease areas of the brain may then be compared to the SUV of a reference region, such as the cerebellum where pathological change is typically absent. The second general approach for analyzing PET scans involves voxel-based morphometry which is used to examine brain change patterns and/or neurodegenerative disease in PET scans. The statistical parametric mapping (SPM) program has become widely used in this analysis. The SPM program scales the image with reference to a cerebral global mean (CGM). Proportional scaling with normalization to the CGM signal is the emblematic voxel-based parameter for analyses of dementia in the brain. Either the cerebellum or primary sensorimotor cortex are typically selected as reference points for the analysis.

Many analysis methods for imaging brain pathology rely on spatial (e.g., 3-D volume) representations of radiopharmaceutical distribution in the brain. For example, the spatially-based image display methods for PET scans provide a convenient means for visualizing the relative amount of pathologic target in three dimensional space. However, the translation of this volumetric PET scan data into a quantitative measure of total pathology (e.g., amyloid burden) in the brain is not readily performed.

The imaging procedure may provide one or more images of volumes of interest in the patient, and in aspects of the invention in which imaging provides more than one image, the multiple images may be combined, overlaid, added, subtracted, color coded or otherwise fused and/or mathematically manipulated by any method known in the art. The image produced may be a digital or analog image that may be displayed as a “hard” image on, for example, printer paper, photographic paper or film or as a digital image or a scanned analog image on a screen, such as, for example, a video or LCD screen.

A variety of image analysis tools or programs known in the art may be used to derive histogram data from brain scan image data. Because digital image data may be necessary for some image analysis tools or programs, in certain aspects of the present invention, a digital image may be prepared from an analog image by, for example, scanning the image prior to image analysis. There are numerous software packages that may be utilized for obtaining a histogram from the imaged brain such as, for example, MRIcroN, MATLAB® R2006B, Statistical Parametric Mapping (SPM), Microsoft Excel and combinations thereof, each which contain a histogram tool capable of converting image data into a histogram of a digital image. Such analysis programs utilize various types of data and methodology to derive a histogram, and the type of data and methodology used may depend upon the specific analysis tools or program. For example, in some aspects of the invention, the analysis program may produce a histogram based on voxel bin numbers and voxel intensities, such as the histograms presented in FIGS. 1A, 1B, 2A, 2B, 3A, 3B, 4A-D and FIGS. 5A and 5B. In other aspects of the invention, the histogram produced may be based on pixel number and pixel intensity in a given plane of the brain. Any such method for producing a histogram may be used in various aspects of the invention.

The radiopharmaceutical used in certain aspects of the invention may be any molecule with an affinity for a compound associated with dementia, VaD, AD, DLB, PD or other neurodegenerative disease. Such radiopharmaceuticals may include, but are not limited to, a radiolabeled antibody, protein, peptide, nucleic acid, organic molecule, small molecule, polymer or a combination thereof. In particular, in some embodiments of the invention, small molecule radiopharmaceuticals may be utilized for radiopharmaceutical imaging, due to the greater degree of diffusability of small molecules, which allows for improved crossing of biological membranes such as the blood-brain barrier, relative to other proteins or polymeric materials.

Aspects of the present invention may be carried out using any radiopharmaceutical useful in imaging β-amyloid or β-amyloid plaque including, but not limited to, those radiopharmaceuticals described in WO 2006/014381 (PCT/US/2005/023617), US 2003/0236391 (Ser. No. 10/388,17), US 2005/0043523 (Ser. No. 10/645,847), WO 2007/047204 (PCT/US2006/039412), WO 2007/086800 (PCT/SE2007/000068), WO 2006/057323, EP 1815872 (PCT/JP2005/021642), WO 2005/016888, EP 1655287 (PCT/JP04/11546), U.S. Pat. No. 6,696,039, U.S. Pat. No. 6,946,116, U.S. Pat. No. 7,250,525, WO 2006/078384 (PCT/US2005/045683), WO 2006/066104 (PCT/US2005/045682), WO 2007/126733 (PCT/US2007/007400), US 2006/269473, US 2006/269474, US2005/0271584, US 2007/0031328, Mathis et al., J Med. Chem. 2003, 46: 2740-2754; Small et al., N Engl. J. Med. 2006, 355: 2652-2663; Zhang et al., Nucl. Med. Biol. 2005, 32: 799-809; Ono et al., Nucl. Med. Biol. 2002, 29:633-642; Ono et al. Nucl. Med. Biol. 2005, 32: 329-335; Qu et al., Bioinorg. Med. Chem. Lett. 2007, 17: 3581-3584; Kemppainen et al., Neurology 2007, 68: 1603-1606, Pike et al., Brain 2007, 130; 2837-2844; Klunk et al., Ann. Neurol. 2004; 55, 306-319; Verhoeff et al., Am J Geriatr Psychiatry 2004; 12, 584-595; and Newberg et al., J Nucl Med. 2006; 47, 748-754, each of which is hereby incorporated by reference in its entirety to the extent such references are not inconsistent with the explicit teachings of this specification.

Further, the radiopharmaceuticals useful in various aspects of the invention may be labeled with any radioisotopes capable of being imaged with a PET or SPECT camera. For example, the radiopharmaceuticals of various aspects may be radiolabeled with radioisotopes, such as, but not limited to, ⁷⁶Br, ¹²³I, ¹²⁵I, ¹³¹I, ^{99m}Tc, ¹¹C, ¹⁸F, other gamma- or positron-emitting radionuclides or a combination thereof. The radioactive half-lives of the radiopharmaceuticals vary, depending on which radioisotope is attached to the compounds. For example, the β-amyloid binding radiopharmaceutical ¹⁸F-AV-45 ((E)-4-(2-(6-(2-(2-(2-[¹⁸F]fluoroethoxy)ethoxy)ethoxy)pyridin-3-yl)vinyl)-N-methylbenzenamine) utilized in the PET image histograms of FIGS. 1A, 1B, 2A, 2B, 3A, 3B, 4A-D and FIGS. 5A and 5B has a radioactive half-life of about 2 hours. Additionally, the amount of radioactivity emitted by the radiopharmaceutical may vary among different aspects of the histogram methods described depending on various aspects of the procedure such as, for example, the waiting period or the physiology of the patient. The precision of measurements and the quality of PET or SPECT images taken when a low dose of the radiopharmaceutical is administered may deteriorate and the time required for imaging the radiopharmaceutical will increase at lower injected doses.

Administration of one or more radiopharmaceuticals may be carried out by any method known in the art, and the radiopharmaceuticals may be administered systemically or locally. For example, in certain aspects of the present invention, one or more radiopharmaceuticals are administered parenterally by, for example, intravenous injection, intramuscular injection or subcutaneous injection, intraperitoneally, buccal administration, or nasal spray. In other aspects, one or more radiopharmaceuticals are administered by bolus injection or infusion. In addition, in some aspects, one or more radiopharmaceuticals are administered systemically using, for example, intravenous injection, and in other aspects, one or more radiopharmaceuticals are administered locally by, for example, injection into the brain or the carotid artery.

In certain aspects of the invention, a waiting period may follow the administration of one or more radiopharmaceuticals. For example, following administration, the amyloid plaque-specific radiopharmaceutical may be imaged as long as the total procedure time is of reasonable duration for the patient. Aspects of the invention are not limited by the waiting time, which may range from, for example, about 15 minutes to about 24 hours.

### Examples

In order that the invention disclosed herein may be more efficiently understood, the following examples are provided. These examples are for illustrative purposes only and are not to be construed as limiting the invention in any manner.

### Example 1

### Evaluation of PET Image Histograms for the Differentiation or Diagnosis of Subjects with Alzheimer's Disease Pathology versus Normal Control Subjects

The histogram and histogram data were generated using the MRIcroN histogram application. The MRIcroN program was applied to selected PET scan images to determine the number of voxels (bin count on the y-axis) at a given voxel (or pixel) intensity value (x-axis) for each scan. A histogram was generated for each PET scan wherein the intensity level (on the x-axis) corresponds to the concentration of amyloid plaque (represented by radioactivity present in a given voxel). Pixel/voxel intensities ranged from 0-256 (2⁸) for gray scaled images. For example, if a histogram displayed a value of 3000 on the y-axis for a value of 130 on the x-axis it was concluded that 3000 voxels had an intensity of 130.

Evaluation of the PET image histograms of ¹⁸F-AV-45 (an amyloid-plaque binding radiopharmaceutical) revealed two distinct histogram forms. One histogram form characterized a patient with Alzheimer's disease (AD) while the other histogram form was indicative of a healthy control (HC) subject. Each of the histograms were analyzed and compared by moving left to right on the x-axis. For histograms created from the image of the whole head (i.e., including extra-cranial and intra-cranial radioactive signal) a first “off-scale” peak at low pixel/voxel intensity values on the x-axis represents activity outside the brain. Each such histogram also contains a second peak at intermediate pixel/voxel intensity values on the x-axis that correlates to natural or healthy regions of the brain (containing no amyloid plaque). The amyloid plaque-positive AD patient histogram displays an additional peak at higher pixel/voxel intensity values representing an elevated amount of unhealthy β-amyloid plaque formation throughout the brain. Conversely, the histograms of the healthy control subjects display smaller y-axis values approaching 0 at the same region on the x-axis.

To exploit the differences in the histograms of the AD patients and the healthy control patients, an Area Under the Curve (AUC) analysis was performed using the data values from MRIcroN. After examining the AD patient histograms, it was observed that the high-intensity peak consistently resided within the 125-200 pixel/voxel intensity region on the x-axis for this specific analysis. Based on this observation, an AUC analysis of all histograms was performed in the 125-200 pixel/voxel intensity region on the x-axis using the trapezoidal approximation method. The trapezoidal approximation method uses a summation of small trapezoid areas. The formula for this method is Σ(y₁+y²)/2*(x₁−x₂) for which x₁ and x₂ each increase incrementally by 1 starting at 125, 126 and ending at 199, 200 (y: y-axis values, x: x-axis values). After a comparison of the areas under the curves within the 125-200 pixel/voxel intensity region on the x-axis of each of the histograms, it was observed that there was a substantial difference between the areas of the histograms of the AD patients and the histograms of the healthy control patients with respect to this higher intensity (i.e., third peak) region. Table 1 lists AUC analysis data within the 125-200 pixel/voxel intensity region on the x-axis. As shown in Table 1, the AD patient histograms, on average, had an area under the third peak region that was 1837.6 units higher than same region of the histogram of the healthy control subjects.

TABLE 1
Area Under the Curve (AUC) Analysis of Alzheimer's Disease
(AD) Histograms and Healthy Control (HC) Histograms over the
Third Peak (125-200 pixel/voxel intensity region) on the x-Axis.

| | Total Area Under |
|---|---|
| Patient | Second Curve |
| 12_18B-AD | 4349.5 |
| 12_08B_AD | 3116.5 |
| 12-13B-AD | 2463 |
| 12_15B-AD | 2436 |
| 12_21B-AD | 2252.5 |
| 23_08B-HC | 544 |
| 23_05B-HC | 593 |
| 12_04A-HC | 1482.5 |
| 23_01B-HC | 932 |
| 26_07B_HC | 1878 |
| HC Averages | 1085.9 |
| AD Averages | 2923.5 |
| Average | 1837.6 |
| Difference | |

### Example 2

### Measurements of the Relative Amount of Amyloid Plaque in Normal Subjects and Subjects with Amyloid Pathology Utilizing a Derivative Curve Separation Method Applied to the Image Histogram

¹⁸F-AV-45 brain PET scans were analyzed using histograms of amyloid-pathology-negative and amyloid-pathology-positive patients. The separation of the lower intensity, non-specific radiopharmaceutical voxel bins from the higher intensity voxel bins was accomplished by identifying the inflection point on a descending slope of a histogram curve (based on a first derivative analysis), which enabled the histogram to be divided into two parts, as shown in FIG. 6. FIG. 7 is a graph of the ratios of the areas under the histogram curve for post-inflection point (i.e. higher intensity specific radiopharmaceutical binding) relative to pre-inflection point (i.e. lower intensity non-specific radiopharmaceutical binding) voxel populations from the amyloid PET scans of cognitively normal and Alzheimer's disease subjects (red line separates both groups).

Although the present invention has been described in considerable detail with reference to certain preferred aspects thereof, other versions are possible. Therefore, the spirit and scope of the appended claims should not be limited to the description and the preferred versions contained within this specification.

What is claimed is:

1. A method for detecting the presence of a pathologic target in a brain of a patient comprising:
administering to a patient a radiopharmaceutical capable of binding to a pathologic target in the brain of the patient;
obtaining image data of at least a portion of the cranium of the patient;
generating a histogram from the image data; and
analyzing a feature of the histogram to detect the presence of the pathologic target in the patient's brain.

2. The method of claim 1, wherein the histogram represents the entire image volume from the image data of the cranium.

3. The method of claim 2, wherein the histogram includes more than two peaks or a dispersed distribution of elevated higher intensity bin counts over a segment of the histogram, thereby indicating the presence of the pathologic target in the patient's brain.

4. The method of claim 2, wherein the histogram includes two peaks representing relatively lower intensity radioactivity in the patient's cranium compared to a histogram for a comparative patient having a significant amount of the pathologic target in the comparative patient's cranium, thereby indicating the absence of significant pathologic target in the patient's brain.

5. The method of claim 1, further comprising the step of eliminating from the image data radioactive signal from outside of the patient's brain.

6. The method of claim 5, wherein the histogram includes two or more peaks or a dispersed distribution of elevated higher intensity bin counts over a segment of the histogram, thereby indicating the presence of the pathologic target in the patient's brain.

7. The method of claim 5, wherein the histogram includes one peak representing relatively lower intensity radioactivity in the patient's cranium compared to a histogram for a comparative patient having a significant amount of the pathologic target in the comparative patient's cranium, thereby indicating the absence of significant pathologic target in the patient's brain.

8. The method of claim 1, wherein the pathologic target is an abnormal concentration of a native or pathologically-altered protein, peptide or oligonucleotide.

9. The method of claim 1, wherein the pathologic target is β-amyloid aggregates.

10. The method of claim 1, wherein the pathologic target is α-synuclein aggregates.

11. The method of claim 1, wherein the pathologic target is phosphorylated-tau protein aggregates.

12. The method of claim 1, wherein the pathologic target is Lewy bodies.

13. The method of claim 1, wherein the pathologic target is neurofibrillary tangles.

14. The method of claim 1, wherein the pathologic target is micro-thrombi in the vascular system of the patient's brain.

15. The method of claim 1, wherein the pathologic target is a tumor cell-associated antigen in the patient's brain.

16. The method of claim 1, wherein the pathologic target is an antigen that is upregulated due to inflammation in the patient's brain.

17. The method of claim 1, wherein the pathologic target is an antigen which is upregulated due to vascular disease in the patient's brain.

18. The method of claim 1, wherein the step of obtaining image data includes using positron emission tomography (PET) imaging, single photon emission tomography (SPECT) imaging, PET with concurrent computed tomography imaging (PET/CT), PET with concurrent magnetic resonance imaging (PET/MRI) or a combination thereof.

19. The method of claim 1, wherein the histogram is derived from voxel intensity and frequency data extracted from the image data.

20. The method of claim 1, wherein the histogram is derived from voxel-by-voxel or pixel-by-pixel data extracted from the image data.

21. The method of claim 1, wherein the analyzing step includes analyzing a feature of the histogram including at least one one of the following: shape, area, peak value, and frequency.

22. The methods of claim 1, further comprising the step of measuring absolute or relative area of the histogram.

23. The methods of claim 1, further comprising the step of measuring peak intensity for specific and non-specific binding sites for the radiopharmaceutical in the patient's brain.

24. A method for analyzing a brain image histogram for the detection or measurement of a pathologic target in a brain of a patient comprising:
administering to a patient a radiopharmaceutical capable of binding to a pathologic target in the brain of the patient;
obtaining image data of the brain of the patient;
generating a histogram from the image data, wherein the histogram includes intensity versus frequency curves resulting from different populations of binding sites of the radiopharmaceutical in the patient's brain; and
applying a mathematical method of separating or deconvoluting the intensity versus frequency curves.

25. The method of claim 24, wherein the step of separating or deconvoluting the intensity versus frequency curves includes derivative or Fourier transformations of the data.

26. The method of claim 24, further comprising the step of fitting the histogram using a method selected from Gaussian fitting, exponential fitting, polynomial fitting, and combinations thereof.

27. A method for detecting neurodegenerative disease in a patient comprising:
administering at least one radiopharmaceutical for detecting a pathologic target associated with a neurodegenerative disease to a patient;
measuring the distribution of radioactivity of the at least one radiopharmaceutical in a region of the cranium of the patient wherein the pathologic target associated with the neurodegenerative disease is anticipated to be positioned;
employing computer executable logic to manipulate the measured distribution of radioactivity of the radiopharmaceutical to generate a histogram of intensity and frequency; and
analyzing the characteristics of the histogram to detect and quantitate the presence of the pathologic target associated with the neurodegenerative disease.

28. The method of claim 27, wherein the pathologic target is neuritic plaques (NPs).

29. The method of claim 27, wherein the step of measuring the distribution of radioactivity comprises positron emission tomography (PET) imaging, single photon emission tomography (SPECT) imaging, PET with concurrent computed tomography imaging (PET/CT), PET with concurrent magnetic resonance imaging (PET/MRI) or a combination thereof.

30. The method of claim 27, wherein the histogram represents the entire image volume from the image data.

31. The method of claim 27, wherein the histogram is derived from voxel-by-voxel data extracted from the measured distribution of radioactivity of the radiopharmaceutical.

32. The method of claim 27, wherein the neurodegenerative disease is at least one of dementia, cognitive impairment, Alzheimer's disease (AD), Parkinson's disease (PD), Dementia with Lewy Bodies (DLB), Vascular Dementia (VaD), and combinations thereof.

33. A method for predicting the relative risk for future development of a dementia such as Alzheimer's disease (AD) or Dementia with Lewy Bodies (DLB) or Vascular Dementia (VaD) in a subject comprising:
administering to the subject one or more radiopharmaceuticals that bind to aggregates of β-amyloid, α-synuclein, tau protein or micro-thrombi in the subject's brain;
obtaining image data of the subject's brain;
generating an intensity versus frequency histogram from volume elements of the image data; and
evaluating at least one of shape, area, peak value and graphical parameters of the histogram to detect the presence of the aggregates of β-amyloid, α-synuclein, tau protein or micro-thrombi in the subject's brain, wherein the absence of a higher intensity peak in the histogram due to specific binding of the radiopharmaceutical to the pathologic target within the subject's brain is indicative of lower risk of future development of the dementia, and the presence of the higher intensity peak in the histogram due to the specific binding of the radiopharmaceutical to the pathologic target within the subject's brain is indicative of a relatively higher risk for the presence or future development of the dementia.
